(19) United States
(12) Patent Application Publication
(10) Pub. No.: US 2005/0196778 A1
(43) Pub. Date: Sep. 8, 2005

(54) Nucleic acid analysis apparatus

(76) Inventors: Shuhei Yamamoto, Hitachinaka (JP); Muneo Maeshima, Mito (JP); Katsunobu Hama, Hitachinaka (JP); Satoshi Takahashi, Hitachinaka (JP)

Correspondence Address: DICKSTEIN SHAPIRO MORIN & OSHINSKY LLP, 2101 L Street, NW, Washington, DC, 20037, US

(21) Appl. No.: 11/013,403

(22) Filed: Dec. 17, 2004

(30) Foreign Application Priority Data
Dec. 19, 2003 (JP) 2003-423410

(51) Int. Cl. C12Q 1/68; C12M 1/34

(52) U.S. Cl. 435/6; 435/287.2

(57) ABSTRACT

An apparatus measures individual measurement sites on a DNA chip in a short period of time. The DNA chip is irradiated by light-emitting diodes (LEDs) so as to excite fluorescent dye at each measurement site, and fluorescence emitted from the individual measurement sites is detected all at once. Since substantially uniform measurement conditions can be obtained for each measurement site, measurement accuracy increases. The read mechanism requires less space and is less costly, thereby decreasing the failure rate and virtually eliminating the need for maintenance of the apparatus.

### BACKGROUND OF THE INVENTION

1. Field of the Invention

The present invention relates to nucleic acid analysis apparatuses, such as an SNP analysis apparatus for analyzing single nucleotide polymorphism (SNP) of a purified DNA sample.

2. Description of Related Art

JP Patent Publication (Kokai) No. 2000-131237 A discloses a conventional method and apparatus for reading DNA array chips. The disclosed technology involves moving a DNA chip and an excitation light source relative to each other, scanning the DNA chip using a laser beam of light, and detecting fluorescence with a photomultiplier (PMT), thus reading the DNA chip.

### SUMMARY OF THE INVENTION

In the conventional reading technique employing a laser oscillator as the excitation light source and a photomultiplier as the detector, only information at individual points on the DNA chip is obtained. Thus, in order to obtain two-dimensional information about the DNA chip, a mechanism is required for scanning inside the DNA chip. Furthermore, if there are two kinds of fluorescent dye as the object of measurement, two sets of laser oscillators and photomultipliers are required. Thus, in order to read the DNA chip, each measurement site on the DNA chip must be scanned, and, for each measurement site, the light sources and detectors must be switched for measurement as many times as the number of the fluorescent dyes that must be detected, resulting in a longer measurement time. The ever-increasing degree of integration of the DNA chips is also another factor contributing to the extension of measurement time.

It is therefore an object of the invention to enable each measurement site on a DNA chip to be measured within a short time.

Specifically, in accordance with the invention, a DNA chip is irradiated with a light-emitting diode (LED) to thereby excite the fluorescent dye at each measurement site thereon, and the fluorescence emitted from individual measurement sites is detected using a CCD camera in a collective manner.

In accordance with the invention, individual measurement sites on the DNA chip can be measured all at one time. The measurement conditions at each measurement site can be made substantially identical, leading to an improvement in measurement accuracy. The size, cost, and the failure rate of the reading mechanism can be reduced, and virtually no maintenance is required.

### BRIEF DESCRIPTION OF THE DRAWINGS

FIG. 1 shows a broken perspective view of an SNP analysis apparatus employing a DNA chip.

FIG. 2 schematically shows the SNP analysis apparatus.

FIG. 3 schematically shows a cross section of an optical system unit.

FIG. 4 shows an external view of the optical system unit.

FIG. 5 shows LEDs in detail.

FIG. 6 shows a cross section of a lens unit and a side view thereof.

FIG. 7 illustrates how a detection filter is switched and confirmed.

FIG. 8 shows a flowchart of processes.

### DETAILED DESCRIPTION OF THE PREFERRED EMBODIMENTS

The nucleic acid analysis apparatus according to the invention is described hereafter by way of an SNP analysis apparatus as a particular example thereof, with reference made to the attached drawings, which are for illustration purposes only and do not limit the scope of the invention.

The nucleic acid analysis apparatus of the invention is suitable for analyzing those semiconductor chips called nanochips, as well as the general DNA chips such as, for example, those DNA chips manufactured by Affymetrix, Inc. Nanochips are semiconductor chips on which electrodes are disposed in a matrix whose surface is coated with a transmitting layer structure. Users can construct oligonucleotide arrays as desired, spike them with reagents, and analyze a PCR product, which is the sample. In the case of general DNA chips, DNA chip manufacturers supply DNA chips equipped with oligonucleotide arrays having known base sequences. These DNA chips comprise a glass substrate or the like on which four kinds of bases (A, G, C, T), which are precursors of oligonucleotide, are disposed at predetermined locations, thereby forming a oligonucleotide array. Namely, the oligonucleotide is extended on the glass substrate to form the oligonucleotide array. On the other hand, in the case of nanochips, a semiconductor chip whose surface is covered with a transmitting layer structure of agar is used, wherein a nucleic acid that is prepared in advance is immobilized in the transmitting layer structure to thereby form a nucleic acid array.

### Embodiments

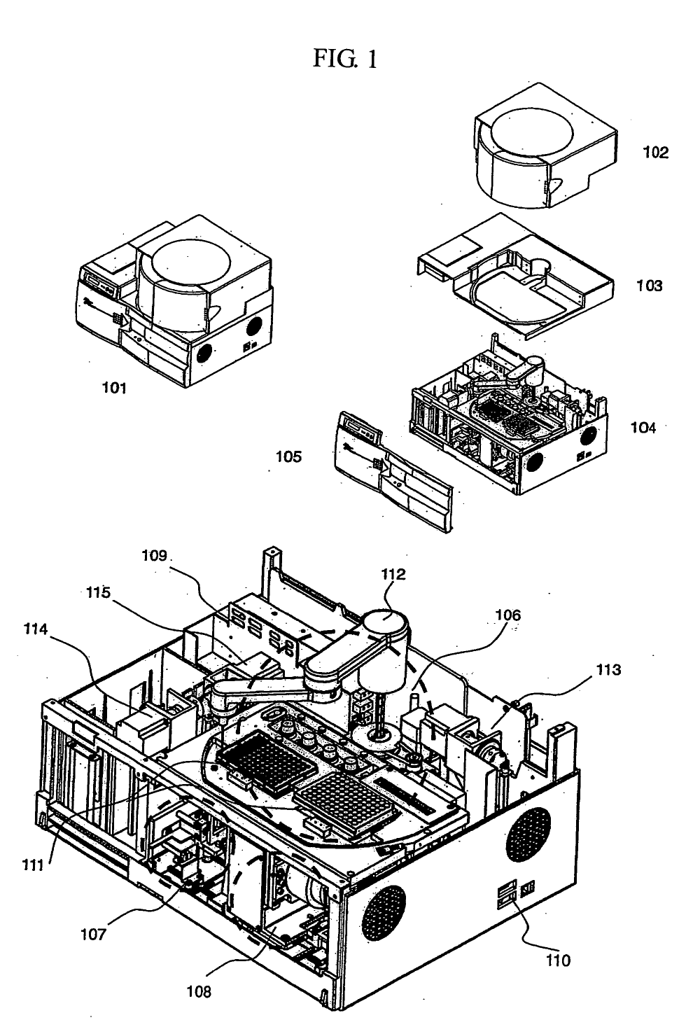

FIG. 1 shows a broken perspective view of a SNP analysis apparatus in which a DNA chip in accordance with an embodiment of the invention is utilized. The outline of the SNP analysis apparatus is described with reference to FIG. 1.

SNP analysis apparatuses are mainly used for detecting SNPs of DNA. The SNP stands for single nucleotide polymorphism, and it refers to those genes that differ from one another in only one base. The SNPs are thought to be related to diseases, and searches are vigorously conducted for SNPs related to various diseases.

The SNP analysis apparatus of the embodiment provides a final output in the following three states: states in which an SNP of interest exists, or does not exist, in a sample DNA in both alleles (“homo”), and a state in which an SNP of interest exists in only one of the alleles (“hetero”). In an example of method for distinguishing among them, a reagent that reacts with and binds to (hybridizes with) a sample having an SNP of interest is labeled with a first fluorescent dye, and a reagent that hybridizes with samples not having the SNP of interest is labeled with a second fluorescent dye which differs from the first fluorescent dye in excitation wavelength and fluorescent wavelength. The both reagents are caused to react with the sample DNA. Fluorescence from the first and the second fluorescent dyes is detected, and their detection intensity ratio is determined, based on which the aforementioned three kinds of states are distinguished with respect to the SNP of interest.

A procedure for analyzing the PCR product as the sample using the aforementioned nanochip is described. The analysis procedure has two kinds of formats, namely an amplicon down format and a capture down format.

In the amplicon down format, an analysis target PCR product (sample oligos) with a partially unknown base sequence and with a biotinylated end is supplied onto a semiconductor chip, and a voltage is applied to a certain electrode on the semiconductor chip. The sample oligos are then drawn towards the electrode and come into contact with the transmitting layer structure on the semiconductor chip surface. The biotin label of the sample oligos reacts with the transmitting layer structure (avidin-biotin reaction) such that the sample oligos are immobilized on the transmitting layer structure. The semiconductor chip surface is then washed, and the above steps are repeated using another sample oligos, thereby forming a desired oligonucleotide array consisting of the sample oligos on the semiconductor chip. After the desired oligonucleotide array is formed in which the sample oligos are disposed in a matrix, an oligonucleotide (reporter oligos) with a fluorescent-labeled end is supplied thereon. The reporter oligos hybridize with the sample oligos having a complementary sequence. After the semiconductor chip surface is washed, the semiconductor chip is irradiated with excitation light, causing the reporter oligos that have hybridized with the sample oligos to emit fluorescence. By detecting and analyzing the fluorescence pattern, the base sequence of the sample oligos can be analyzed.

On the other hand, in the capture down format, an oligonucleotide (capture oligo) having a known base sequence and with a biotinylated end is supplied onto a semiconductor chip, and a voltage is applied to a certain electrode on the semiconductor chip. The capture oligo is then drawn to the electrode and comes into contact with the transmitting layer structure on the semiconductor chip surface. The biotin label of the capture oligo reacts with the transmitting layer structure (avidin-biotin reaction), such that the capture oligo is immobilized on the transmitting layer structure. The semiconductor chip surface is then washed, and the above steps are repeated using another capture oligo, thereby forming a desired oligonucleotide array consisting of the capture oligo on the semiconductor chip. After the oligonucleotide array in which the capture oligo is disposed in a matrix is formed, an analysis target PCR product (sample oligos) with a partially unknown base sequence is supplied thereon. The sample oligos hybridize with the capture oligo having a complementary sequence, such that the sample oligos are immobilized on the semiconductor chip via the capture oligo. The semiconductor chip surface is then washed, and an oligonucleotide with a fluorescent-labeled end (reporter oligos) is supplied. The reporter oligos hybridize with sample oligos having a complementary sequence. The semiconductor chip surface is then washed, and the semiconductor chip is irradiated with excitation light, thereby causing those reporter oligos that have hybridized with the sample oligos to emit fluorescence. By detecting and analyzing the fluorescence pattern, the base sequence of the sample oligos can be analyzed.

The SNP analysis apparatus is mainly made up of a dispensing unit, a flow passage system unit, an interface unit, an optical system unit, and a power supply unit, and is controlled by external equipment (PC), which is not shown. The SNP analysis apparatus is adapted such that a cartridge equipped with a DNA chip can be attached thereto for analysis. The cartridge contains a flow cell in which a DNA chip consisting of a semiconductor element is disposed. The DNA chip is capable of accommodating a maximum of 400 nucleic acid probes at predetermined locations in order to construct a desired probe array. The apparatus is capable of automatically performing the series of steps from attaching the nucleic acid probes onto the DNA chip up to measurement, so that there is no need for the operator to manipulate the apparatus for each sample. For example, an analysis of 96 samples can be performed in approximately three hours.

The SNP analysis apparatus (101) includes a top cover (102), a cover (103), a main body (104), and a front panel (105).

The dispensing unit includes a sample handling station (106) capable of arranging and storing samples and reagents, and a robot arm (112) for transporting samples and reagents to predetermined positions. The sample handling station (106) can mount two sample trays (111) containing samples and reagents, and four reagent bottles. The sample handling station (106) also includes a reacting enzyme cooling portion capable of storing reacting enzymes in a refrigerated state. Using the robot arm (112), which is equipped with a probe tip for sucking and discharging a solution, a predetermined sample or reagent can be dispensed and put into an injection port.

The flow passage system unit is capable of actuating three syringe pumps in order to automatically carry the sample or reagent that has been put in through the injection port, the water stored in a water bottle, and so on, to the DNA chip inside the flow cell. The flow passage system unit is also capable of washing the probe tip via a washing port, as well as the interior of the flow cell and the flow passages. The unit is also capable of discharging waste liquids produced in the apparatus to a waste liquid bottle disposed outside the apparatus.

The interface unit is adapted to retain the cartridge, which is detachable. By inserting needles into the cartridge, the cartridge can be connected to the flow passage system via the needles. Thus, a predetermined sample or reagent can be carried to the flow cell. By providing an electric connection with the DNA chip, the interface unit makes it possible to control the location of the nucleic acid probes, for example. Further, by providing a thermal connection with the DNA chip, the interface unit makes it possible to control the temperature inside the flow cell.

The optical system unit includes a light source for exciting a fluorescent reagent that exists on the DNA chip, and a detector for detecting the fluorescence emitted by the fluorescent reagent, so that the optical system unit can output an image on the DNA chip.

The power supply unit supplies drive power to each unit and is operable with power supply voltages of 100, 110, 120, 200, 220, 230, and 240 VAC. It is adapted for current values of 4 A or less and frequencies of both 50 Hz and 60 Hz.

On the side surface of the SNP analysis apparatus (101), there is provided an external equipment connector (110) allowing connection with a personal computer (PC) and a bar-code reader, which are not shown. The PC is used for the operation of the SNP analysis apparatus (101) and for the display, analysis and storage of measurement results. The SNP analysis apparatus (101) and the PC are connected via Ethernet. By using a hub, it becomes possible to operate up to four SNP analysis apparatuses (101) with one PC. The bar-code reader reads a bar code affixed to the sample tray containing a sample or to the cartridge, and then transmits the information to the PC, thereby facilitating the management of the samples, reagents, and cartridge that are measured.

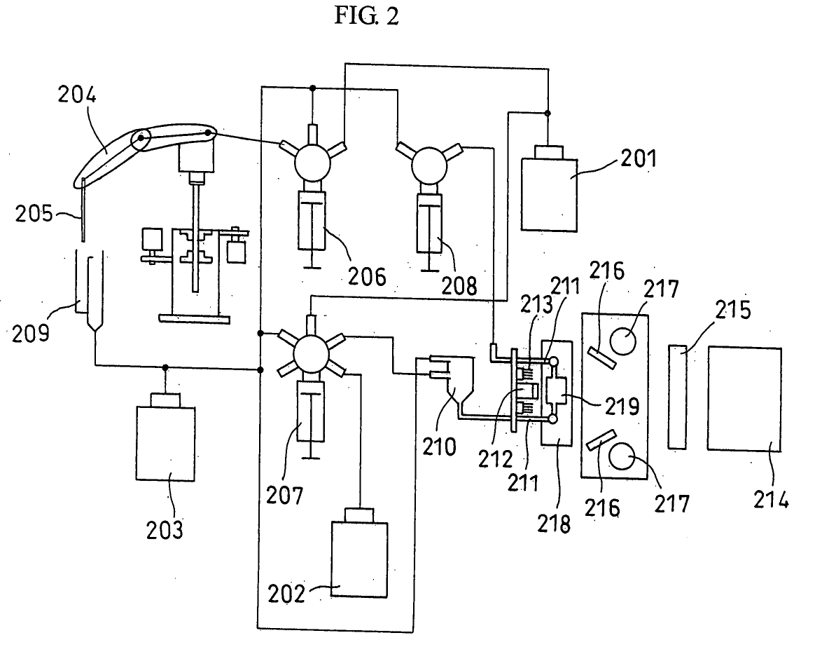

FIG. 2 schematically shows the SNP analysis apparatus according to the present embodiment. The overall mechanical portion of the apparatus is described in detail with reference to FIG. 2, in which the cartridge (218) is inserted into the apparatus main body, which is connected to a flow cell (219) via needles (211).

The flow passage system unit is made up of a plurality of flow passages connecting a probe tip (205) of a robot arm (204), a water bottle (201), a histidine bottle (202), a waste liquid bottle (203), a water pump (206), a histidine pump (207), a cartridge pump (208), a washing port (209), an injection port (210), and needles (211).

The probe tip (205) is connected to a pipe for sucking and discharging a sample or a solution. The tip can be moved to a predetermined position by the robot arm (204). Using the probe tip (205), a predetermined sample or reagent can be dispensed from a sample tray or a reagent bottle disposed on the sample handling station, or from the reacting enzyme cooling portion, and the sample or reagent can then be put into the injection port (210). The injection port (210) is a port tank via which the ample or solution is delivered to the flow cell (219) in the cartridge (218). The injection port (210) can be temperature-controlled in the range between 40° C. and 60° C. If the sample is introduced when the temperature of the introduced sample and the internal temperature of the cartridge are different, a spike noise is generated. The spike noise can be reduced by controlling the temperature of the injection port (210) prior to the introduction of the sample. The probe tip (205) can be washed in the washing port (209).

The water pump (206) can deliver water from the water bottle (201) to the probe tip (205), suck or discharge the sample or reagent through the probe tip (205), and deliver a solution to the waste liquid bottle (203).

The histidine pump (207) can send histidine from the histidine bottle (202) to the injection port (210), send water from the water bottle (201) to the injection port (210), send air to the injection port (210), and send a solution in a syringe to the waste liquid bottle (203).

The cartridge pump (208) can send a solution and air from the injection port (210) to the flow cell (219) inside the cartridge (218), and send a solution and air from the flow cell (219) inside the cartridge (218) to the cartridge pump (208).

The waste liquid produced in the apparatus can be discharged to the waste liquid bottle (203) that is externally provided and detachably connected to the apparatus via the connector portion.

The reagent or washing liquid put in the injection port can be carried, via the cartridge flow passage, to the flow cell (219) by driving the water pump (206) and the cartridge pump (208). The cartridge flow passage can be washed with histidine by driving the histidine pump (207) and the cartridge pump (208). The flow passage from the histidine pump (207) via the injection port (210) to the cartridge pump (208) has such a structure that the histidine solution can be substituted by water. By washing the passageway with water, precipitation of the histidine solution inside the flow passage piping is avoided.

The water bottle (201) and the histidine bottle (202) can hold 1 L of water and histidine, respectively, so that there is no need for the user to add water or histidine between the step of affixing of 96 samples and the step of measurement.

The interface unit includes the needles (211) that constitute a flow passage connecting the flow cell (219) inside the cartridge (218) and the apparatus main body, a cartridge cooling Peltier element (212), and a pogo pin (213). The cartridge cooling Peltier element (212) comes into contact with the cartridge so as to control the temperature of the flow cell (219). The pogo pin (213) is connected to the terminals provided on the cartridge (218) so as to provide electrical connection between the apparatus main body and the DNA chip. The pogo pin (213) has only 12 pins, so that its connection to the cartridge is easy.

The optical system unit includes a CCD camera (214), an EM filter (215), EX filters (216), and LEDs (217). The LEDs (217) are filtered by the EM filter (215) in terms of wavelength characteristics before the fluorescent material on the DNA chip inside the flow cell (219) is irradiated with the light emitted by the LEDs. The fluorescence produced by the fluorescent material is filtered by the EX filters (216) in terms of wavelength characteristics, and an image of the flow cell (219) is captured by the CCD camera (214). The LEDs (217) constitute the light source that has a much longer life than the conventional laser, thereby reducing the frequency of required replacement of the light source during the life of the apparatus and facilitating maintenance.

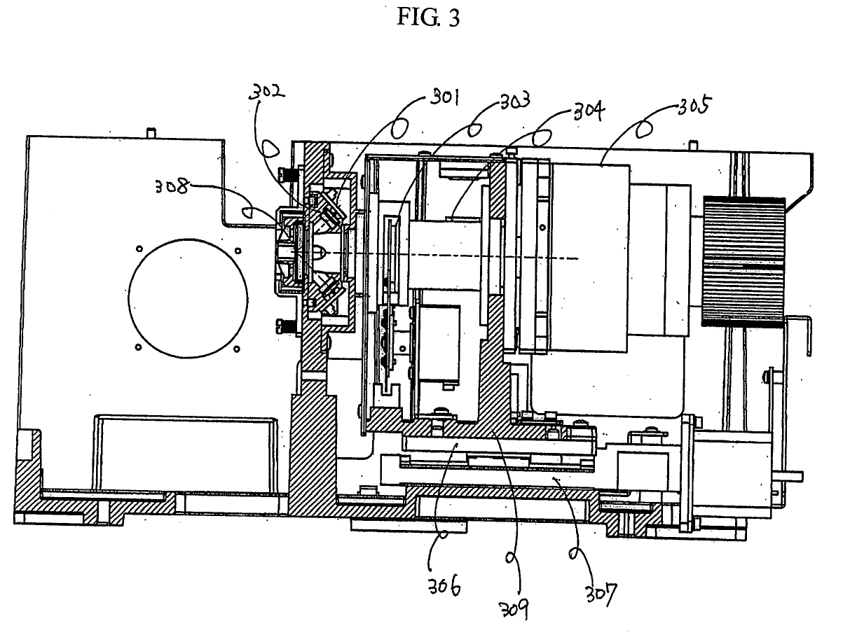

The optical system unit is described in detail. FIG. 3 schematically shows the optical system unit, which includes an LED (301), an excitation filter (302), a detection filter (303), a lens unit (304), a CCD camera (305), a transport stage (306), and an auto-focus driving portion (307).

In order to excite the fluorescent dyes that exist on the DNA chip, a plurality of light-emitting diodes (LEDs) are used, of which there are two types with different emission wavelengths corresponding to the two kinds of fluorescent dyes. The light emitted from the LEDs (301) passes through the excitation filter (302) and then irradiates the entire surface of the DNA chip (308) simultaneously. Fluorescence emitted by the fluorescent dyes distributed on the DNA chip is focused by the lens unit (304) consisting of a plurality of lenses on the detector. In the present embodiment, the lens unit consists of five lenses, and the detection filter (303) is inserted between a first and a second lens. The detection filter that is inserted consists of three kinds of filters that are automatically switched depending on the purpose of measurement. The detector is formed by a charge-coupled device (CCD). The lens unit (303), detection filter (304), and CCD camera (305) are disposed on the transport stage (306) that can be moved in the optical axis direction by the auto-focus drive portion (307), which is made up of a ball screw and a stepping motor and which is moved for focusing purposes.

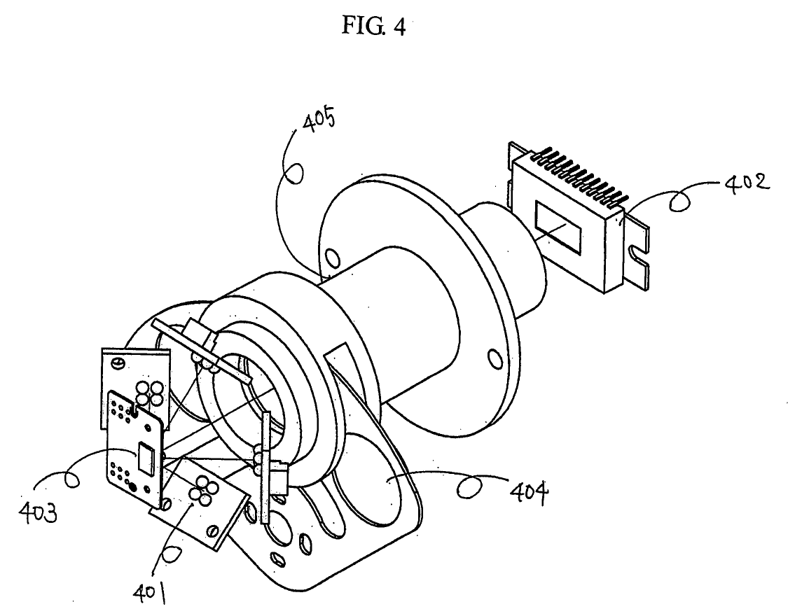

FIG. 4 shows an external view of the optical system. As mentioned earlier, in the present embodiment, the excitation light source is formed by LEDs (401), and the detector is formed by a CCD (402). In accordance with the embodiment, the presence or absence of fluorescent material with a density exceeding a certain level can be measured so as to detect SNPs with known association with diseases and to perform diagnosis of a disease.

In accordance with the conventional technology, a confocal optical system is used that employs a laser oscillator as the excitation light source and a photomultiplier (PMT) as the detector. In this system, individual points of the light source and those of the detector are associated such that the resultant information only concerns individual points on the DNA chip, and in order to obtain two-dimensional information about the DNA chip, a mechanism must be provided to scan inside the DNA chip. Furthermore, because there are two kinds of fluorescent dye as the object of measurement, two sets of laser oscillators and photomultipliers are employed, as in the present embodiment. Therefore, when actually conducting a fluorescence measurement, each and every measurement site on the DNA chip must be scanned, and, for each measurement site, the light source and detector must be switched as many times as there are fluorescent dyes that are to be detected, leading to an extended measurement time. It is generally desirable to increase the level of integration of the DNA chip, to mount as many samples on a single DNA chip as possible, and to read them in a shortest possible period of time. While there are 100 measurement sites on the DNA chips in the aforementioned conventional technique, the apparatus of the present embodiment reads DNA chips that have four times as many, or 400 measurement sites. Thus, if the optical system of the conventional technology were to be employed in the apparatus of the embodiment, the measurement time would quadruple. There is another problem associated with the conventional technique, namely the high cost of laser oscillators and photomultipliers. Furthermore, some laser oscillators do not have sufficient life and have failure rates that are higher than those of other light sources, resulting in a reliability problem.

For these reasons, the present embodiment employs the LEDs (401) for the excitation light source and CCD (402) for the detector, whereby the cost and required space can be reduced while increasing throughput. LEDs are much less expensive than laser oscillators and are very small, so that they contribute to a significant reduction in cost and required space. LEDs last longer than other types of light source including laser oscillators and have an extremely low failure rate, thus requiring hardly any maintenance, such as replacement. Furthermore, since the entire surface of the DNA chip (403) is irradiated with the excitation light emitted by the LEDs (401) simultaneously and detection of fluorescence is also performed on the entire surface of the DNA chip (403) simultaneously, using the CCD (402), a significant reduction in measurement time can be achieved as compared with the aforementioned conventional technique.

Generally, the light emitted by an LED has no directionality, which makes it difficult for the LED to emit light in a desired range by itself. Thus, the excitation light emitted by the LED is focused by a lens on the DNA chip. A phenomenon then occurs in which the intensity of the excitation light on the DNA chip differs depending on the position of the measurement site. As mentioned above, the present apparatus determines the presence or absence of three states of SNP on the basis of the fluorescence intensity ratio with regard to two kinds of fluorescent dye. Thus, if the distribution of intensity of the excitation light emitted by the two kinds of LEDs on the DNA chip differs from one measurement site to another, a problem arises that the fluorescent intensity ratio of the two wavelengths would differ depending on the measurement site even if completely identical samples existed at different measurement sites on the DNA chip. To avoid this problem, in the present apparatus, the excitation light intensity distribution on the DNA chip is stored in advance with regard to each of the LEDs with individual wavelengths, and the obtained measurement result is corrected in accordance with the stored distributions. In this way, the difference in detection intensity caused by the difference in magnitude of excitation light intensities can be canceled.

The excitation light produced by the LEDs would normally be reflected by the DNA chip and would reach the detector, together with the fluorescence to be detected. This reflected component of excitation light constitutes a large background noise against which the weak fluorescence would have to be detected. In accordance with the embodiment, in order to eliminate the reflected component of excitation light, two kinds of band-pass filters with different transmitting wavelength bands against the individual fluorescent dyes are used in combination. Specifically, the light that can pass through a first band-pass filter is not allowed to pass through a second band-pass filter, and vice versa. The excitation filter (302), not shown in FIG. 4, which is the first band-pass filter, has a transmitting wavelength band in the emission wavelength band of the LED (401) and therefore transmits part of the light emitted by the LED. On the other hand, the detection filter (404), which is the second band-pass filter, has a transmitting wavelength band in the fluorescence wavelength band of fluorescent dye and therefore transmits part of fluorescence. The excitation filter (302) is inserted between the LED (401) and the DNA chip (403), and only the wavelength component that has passed through the excitation filter (302) irradiates the DNA chip (403) as excitation light. The detection filter (404) is inserted between the DNA chip (403) and the CCD (402) and it prohibits the transmission of the excitation light reflected on the DNA chip, so that only fluorescence is allowed to pass through the lens unit (405) and focused on the detector, or CCD (402).

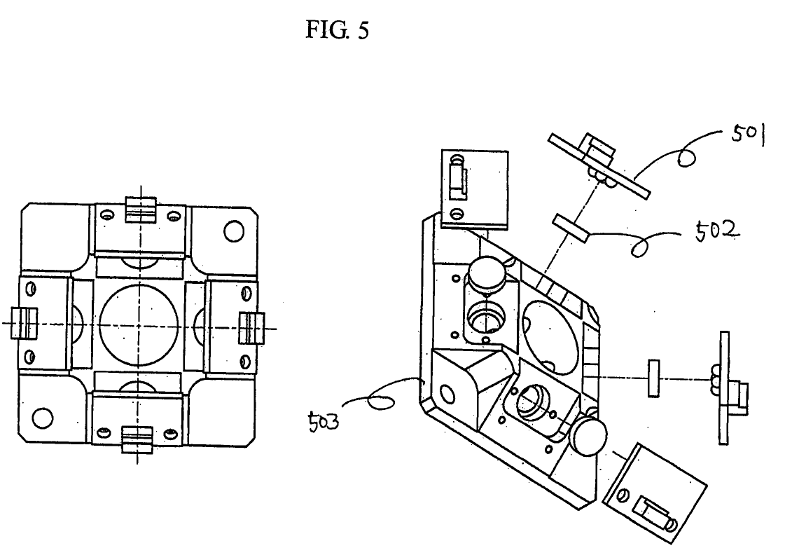

FIG. 5 shows the details of the LED light source portion made up of LEDs (501) and an excitation filter (502). In accordance with the present embodiment, four LEDs with identical emission wavelengths are mounted on a single substrate, and a spherical lens is mounted on each LED. The light emitted by each of the LEDs on the single substrate is focused by the spherical lens on the DNA chip. Two such substrates are provided for each of the two wavelengths, and a total of four substrates are disposed each at a 45° angle with respect to the vertical direction of the DNA chip. The individual substrates are disposed around the DNA chip at 90° intervals such that the substrates of the same wavelength are opposite each other. The multiplicity of LEDs are mounted on each substrate so that the LED light source can be used in combination with the excitation filter. Since the excitation filter (502) used is an interference filter, its transmission characteristics vary depending on the angle of incidence of light. For this reason, the excitation light must be incident on the excitation filter (502) with a certain angle of incidence. However, if the individual LEDs were to be disposed at various positions, for example, it would be necessary to provide an excitation filer for each LED. Accordingly, in order to minimize the number of excitation filters that are required, four LEDs are mounted on each substrate in a close-packed manner, and then one excitation filter is disposed in parallel to each substrate.

Although the excitation filter (502) is designed to exhibit the optimum transmission characteristics when the angle of incidence of light is 90°, some light components are inevitably incident with angles smaller than 90° as long as the light source consists of LEDs. Interference filters have such characteristics that as the incident angle decreases, the transmitting band shifts towards the shorter wavelength side. Thus, the light with an incident angle smaller than 90° contributes only lower wavelength components to the excitation light with which the DNA chip is irradiated, thereby decreasing the excitation light ratio somewhat. Such an influence, however, can be easily compensated for by adjusting the number of LEDs. In the case of the present embodiment, a total of eight LEDs are divided between two substrates, so that no more than two excitation filters are required. Since the transmitting-band shift always appears towards the lower wavelength side, the shift does not cause the transmission band of the interference filter to coincide with that of the detection filter, which would cause the reflected component of excitation light to pass through the detection filter and increase the background noise.

An LED mount (503) for mounting the LEDs (501) and the excitation filters (502) is provided with reference openings for positioning purposes, and the apparatus is provided with corresponding positioning pins. Therefore, the mount (503) can be easily put back to the original position after being detached, so that the irradiation system can be detached and then attached without affecting the excitation light intensity distribution on the DNA chip.

The present embodiment is also provided with the function for adjusting the luminance of excitation light. Specifically, the luminance of the LEDs can be adjusted in 10 levels (between 10% to 100% in 10% steps, with 0% corresponding to the turn-off of LED) by controlling the duty ratio of a PWM signal for controlling the activation of the LEDs. This function is used for controlling the detection intensity of reflected light and fluorescence. Alternatively, the detection intensity may be controlled by adjusting the exposure time of the CCD.

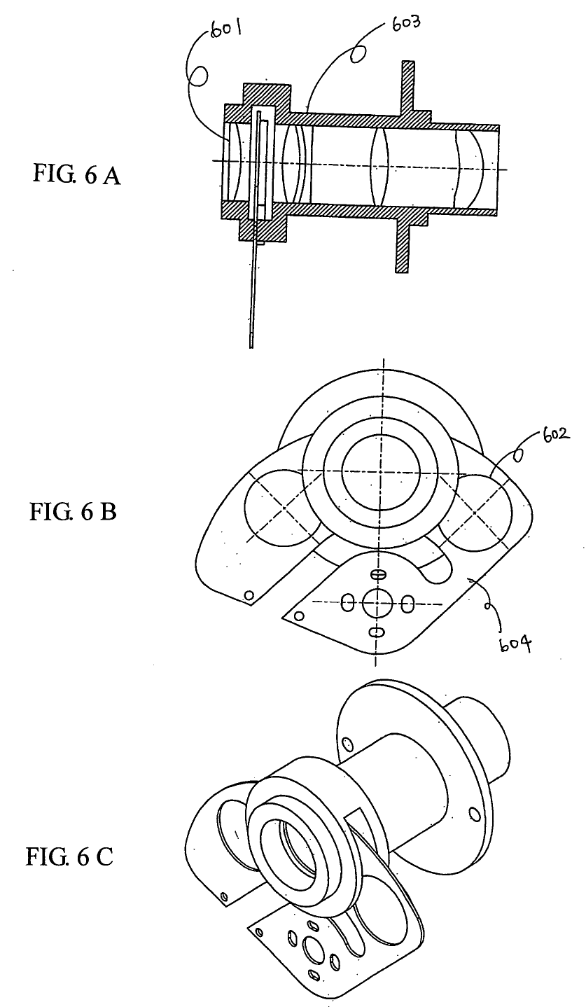

FIG. 6 shows the details of the lens and the detection filter. As shown in FIG. 6(a), the lens (601) used in the present embodiment consists of five lenses, all of which are retained inside a single housing (603), thus facilitating the adjustment of the position of individual lenses with respect to the optical axis and of the interval between the lenses. Furthermore, the housing (603) has a structure that ensures its positional reproducibility when detached from the apparatus and then re-attached thereto, without requiring any adjustments. Thus, once the individual lenses are adjusted outside the apparatus, there is no need to perform further adjustments with regard to the lens positions when the housing (603) is contained in the apparatus.

As shown in FIG. 6(b), the detection filter (602) inserted in the optical path between the DNA chip and the detector has its wavelength characteristics varied by the angle of incidence of light, as in the case of the above-described excitation filter. Therefore, all of the light that is focused on the detector must be caused to be incident on the detection filter at angles within a certain allowable range. For this purpose, the light that leaves the DNA chip and is then incident on the lens is once made into parallel beams by a first lens immediately before the detection filter (602). Since there are three types of detection filters, they must be switched. For this purpose, a through-hole is provided in the housing (603) between the first and second lenses, as shown in FIG. 6(c), and a rotating plate (604) carrying three detection filters (602) is adapted to be rotated in the hole. In this structure, the filter rotating mechanism can be detached and attached without removing the lens housing. The three detection filters (602) consist of two kinds of band-pass filters for detecting fluorescence, and an ND filter that exhibits a constant transmittance in a wide wavelength range. The ND filter is used for obtaining an excitation-light reflected image. The reflected image is mainly obtained when carrying out auto-focusing. The three detection filters are switched by rotating the rotating plate on which they are carried by means of a stepping motor.

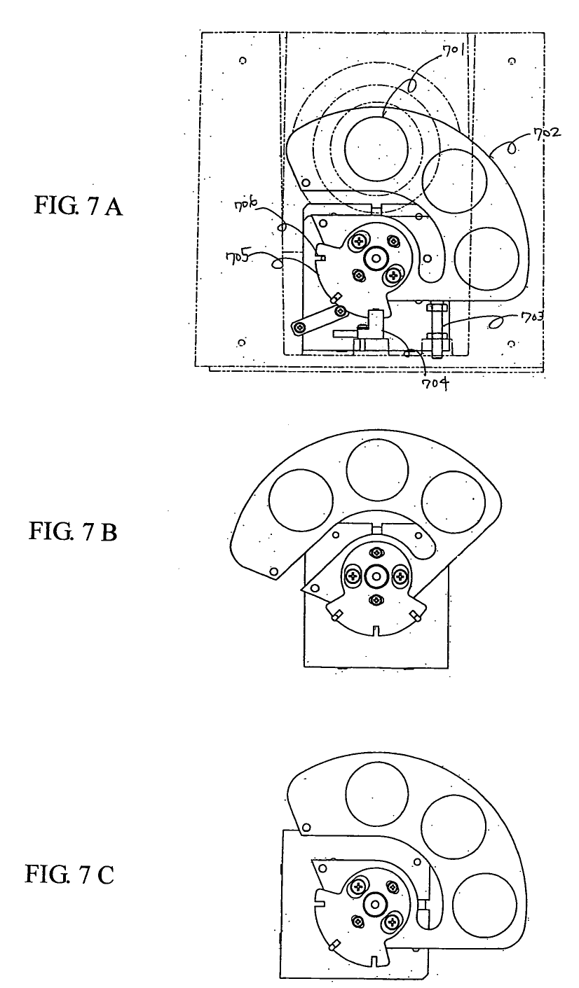

FIG. 7 shows a method of detecting the position of the detection filter. FIGS. 7(a), (b), and (c) each shows the detection filter (701) as actually inserted, seen from the side of the DNA chip.

The reference position of the rotating plate (702) is shown in FIG. 7(a), which position corresponds to the insertion position of the ND filter. The reference position is achieved as the rotating plate abuts a mechanical stopper (703). The stepping motor is rotated a number of times corresponding to the number of pulses required for the rotating plate (702) to reach the mechanical stopper (703) regardless of the instantaneous position of the rotating plate (702). As shown in FIGS. 7(b) and (c), the insertion position for each of the two other band-pass filters can be sequentially reached by rotating the rotating plate (702) from the reference position by 45° intervals.

When the apparatus is used for diagnostic purposes, accurate data cannot be obtained if the fluorescence measurement is conducted without correctly inserting the filter. In a worst case, false diagnosis might be produced. Thus, in order to make sure that the three kinds of filters are correctly inserted, a photointerrupter (704) is used. An additional position detecting rotating plate (705) is mounted on the same rotating axis as that of the rotating plate (702) on which the detection filters (701) are mounted, such that the two rotating plates rotate simultaneously. The three kinds of detection filters (701) are attached around the rotating axis of the rotating plate (702) at 45° intervals. Similarly, slits (706) are provided in the position detecting rotating plate (705) at 45° intervals. The position detecting rotating plate (705) is adjusted such that a corresponding slit (706) is inserted at the position of the photointerrupter (704) when each detection filter (701) is correctly inserted, which causes the photointerrupter (704) to produces an ON output signal. If the detection filter (701) is not inserted at the correct position, the position of the slit (706) is displaced, causing the photointerrupter (704) to produce an OFF signal. Further, in order to deal with the possible case of the photointerrupter (704) producing an ON output at all times due to failure, when switching the detection filter (701), the detection filter (701) is temporarily stopped immediately before it is completely inserted, and then it is confirmed that the output of the photointerrupter (704) is OFF, before the rotating plate (702) is rotated again until a correct insertion position is reached, when it is again confirmed that the output signal from the photointerrupter (704) has turned ON. If it is determined as a result of this procedure that the detection filter (701) has not reached the correct insertion position due to loss of steps in the stepping motor, for example, an error is detected and the apparatus terminates its operation. An error is also detected when the position-confirming photointerrupter (704) is out of order. In this way, the possibility of acquiring erroneous data can be avoided.

The detector is the CCD (402). For reducing noise, the CCD (402) is cooled by a Peltier element. The space surrounding the CCD (402) is air-tightly sealed and is filled with very dry argon gas in order to prevent dew condensation on the CCD due to cooling. As the CCD is of the full-frame type, a mechanical shutter is provided in front of the CCD for the transfer of electric charges.

As shown in FIG. 3, the aforementioned lens unit (304), the mechanism for switching the detection filters (303), and the CCD (305) are all mounted on the same detection system mount (309), which in turn is mounted on the transport stage (306) for the drive system made up of a linear guide, ball screw, and stepping motor. The transport stage (306) is moved primarily for auto-focusing purposes. In the detection-system mount (309), there are provided two reference openings for positioning purposes, as in the above-described LED mount (503). On the transport stage, there are provided reference pins corresponding to the reference openings. By fitting these pins in the openings, the detachment/attachment reproducibility of the detection-system mount can be ensured. By thus disposing the optical components of the detection system, including the lens unit (304), the detection filter switching mechanism, and the CCD (305), on the same component, the entire detection system can be detached and attached easily. As a result, it becomes possible to carry out tasks, such as the replacement of the individual detection-system optical components and position adjustments, outside the apparatus, thereby significantly enhancing the ease of maintenance.

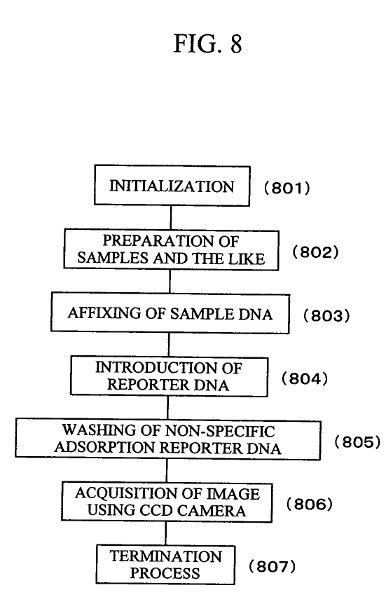

FIG. 8 shows a flowchart of the processes performed by the apparatus. The processes are described with reference to FIG. 8.

After the apparatus is switched on, initialization (801) is performed. The volume of water in the water bottle and the volume of histidine in the histidine bottle are confirmed.

In a preparation of a sample and the like (802), bar codes on sample trays and cartridges containing a low salt buffer, a high salt buffer, NaOH, and a sample are read by a bar-code reader. Each time any of the bar codes is read, an LED lights up, at which location the corresponding item is correctly placed.

A sample DNA is affixed in the following manner (803). The robot probe tip is transported to the washing port where the probe tip is washed by operating the water pump. After the robot probe tip is moved to a target sample position, the water pump is activated so as to suck a sample from the robot probe tip. The robot arm is transported to a PI detection position, and calibration is confirmed. After the robot probe tip is moved to the injection port, the water pump is activated so as to discharge water out of the robot probe tip. The cartridge pump is then activated so as to carry the sample to the cartridge flow cell. An electric current of approximately 0.2 mA is applied to a target position in the cartridge active chip for approximately 60 seconds. The histidine pump is operated so as to deliver histidine to the injection port. The cartridge pump is then activated to move histidine to the cartridge flow cell in order to wash the same. The cartridge pump is activated to send histidine to the waste liquid bottle.

The introduction of a reporter DNA is carried out in the following manner (804). The robot probe tip is moved to the washing port, where the tip is washed by operating the water pump. After the robot probe tip is moved to the position of the high salt buffer, the water pump is activated to suck the high salt buffer from the robot probe tip. After the robot arm is moved to the PI detection position, calibration is confirmed. The robot probe tip is then moved to the injection port, and the water pump is activated to discharge water out of the robot probe tip. The cartridge pump is then activated to transport the high salt buffer to the cartridge flow cell. After the high salt buffer is carried to the cartridge flow cell by activating the cartridge pump, the cartridge pump is activated to carry the high salt buffer to the waste liquid bottle.

The robot probe tip is moved to the washing port, where the probe tip is washed by activating the water pump. After the robot probe tip is transported to the location of the DNA to which fluorescent material is attached (a reporter DNA), the water pump is activated so as to suck the reporter DNA from the robot probe tip. After the robot arm is moved to the PI detection position, calibration is confirmed. After the robot probe tip is moved to the injection port, the water pump is activated so as to discharge water out of the robot probe tip. The cartridge pump is then activated so as to carry the reporter DNA to the cartridge flow cell. The cartridge pump is activated to carry the reporter DNA to the cartridge flow cell, where the reporter DNA is retained for approximately 60 seconds. The cartridge pump is then activated so as to move the reporter DNA to the waste liquid bottle.

The robot probe tip is then transported to the washing port, where the probe tip is washed by activating the water pump. After the robot probe tip is moved to the position of the high salt buffer, the water pump is activated to suck the high salt buffer from the robot probe tip. The robot arm is then moved to the PI detection position, and calibration is confirmed. After the robot probe tip is moved to the injection port, the water pump is activated so as to discharge water out of the robot probe tip. The cartridge pump is then activated to carry the high salt buffer to the cartridge flow cell. The cartridge pump is then activated to carry the high salt buffer to the cartridge flow cell. The cartridge pump is then activated to move the high salt buffer to the waste liquid bottle. The temperature of the cartridge flow cell is raised to a set temperature which is maintained for approximately 60 seconds.

The washing of a non-specific adsorption reporter DNA is carried out as follows (805). The robot probe tip is transported to the washing port, where the probe tip is washed by activating the water pump. After the robot probe tip is moved to the position of the low salt buffer, the water pump is activated to suck the low salt buffer from the robot probe tip. After the robot is moved to the PI detection position, calibration is confirmed. After the robot probe tip is moved to the injection port, the water pump is activated to discharge water out of the robot probe tip. The temperature of the injection port is raised to a set temperature which is maintained for approximately 60 seconds. The cartridge pump is then activated to carry the low salt buffer to the cartridge flow cell. The cartridge pump is then activated to carry the low salt buffer to the waste liquid bottle.

After the robot probe tip is moved to the washing port, the water pump is activated to wash the probe tip. After the robot probe tip is transported to the position of the low salt buffer, the water pump is activated so as to suck the low salt buffer from the robot probe tip. The robot arm is moved to the PI detection position, and then calibration is confirmed. After the robot probe tip is moved to the injection port, the water pump is activated so as to discharge water out of the robot probe tip. The cartridge pump is then activated to carry the low salt buffer to the cartridge flow cell. The temperature of the cartridge flow cell is then raised to a set temperature.

An image is obtained by the CCD camera (806).

The cartridge pump is activated so as to carry the low salt buffer to the cartridge flow cell. The cartridge pump is activated to carry the low salt buffer to the waste liquid bottle. After the apparatus terminating process (807), the apparatus is turned off.

The operation of the apparatus for acquiring an image using the CCD camera is described in detail.

As the cartridge containing the DNA chip is inserted into the apparatus, a specific buffer solution is injected into the flow cell of the cartridge, and then an auto-focusing operation is carried out. During the auto-focusing process, one of the two excitation light sources with different wavelengths is turned on, and the detection filter is switched to the ND filter in order to photograph a reflected image of the DNA chip. This operation is repeated while moving the transport stage at certain intervals. After the edge of the obtained reflected image is enhanced by an image processing, standard deviation of the amount of signal at each pixel in the CCD is calculated, and the resultant value is used as an auto-focusing evaluation value. Larger evaluation values can be interpreted to indicate stronger contrast of the image that is obtained. Thus, the position at which the largest evaluation value is obtained is determined to be the position of focus. In actual operation, the photographing of the reflected image is repeated while moving the transport stage, and at the same time the evaluation value is calculated for each of the images obtained. During the auto-focusing operation, the position of the transport stage and the evaluation value are put into a memory for each of the pictures taken, and the auto-focusing operation comes to an end when the transport stage has moved to the position at which a maximum evaluation value is eventually exhibited. After auto-focusing is completed, samples are affixed to the DNA chip and other preparations are made for the measurement of florescence. Although the auto-focusing operation can be performed at any time, it is desirable to carry it out in the absence of fluorescent dye in order to prevent bleaching, in which the fluorescent dye deteriorates due to optical irradiation. After the samples are affixed to each measurement site on the DNA chip and preparations for fluorescence measurement are completed, a fluorescence image is photographed. Since there are two kinds of fluorescence dye, a fluorescence image is taken for each kind of fluorescence dye. When taking a photograph of a fluorescence image, the detection filter is switched from the ND filter to the band-pass filter for the detection of fluorescence. After the fluorescence image is taken, the fluorescence intensity ratio of the two wavelengths is calculated at each measurement site on the DNA chip, and an SNP determination is made.

In the present embodiment, by using LEDs as the excitation light source and a CCD as the detector, cost and required space can be reduced while achieving higher throughput and easier maintenance over the conventional art. The optical system has adopted a structure where a group of lenses for focusing a DNA chip image on the CCD is housed inside a single housing, and where a plurality of optical filters for the selection of the wavelength of reflected light or fluorescence from the DNA chip can be switched through a though-hole provided in a part of the housing. The structure makes it easier to assemble and adjust the lenses and the filter switching mechanism. Furthermore, the irradiation system and the detection system are allowed to be individually detached and attached within the optical system unit while achieving a high positional reproducibility. Thus, the construction and arrangement of the individual components in the optical system unit can be simplified, thereby facilitating the assembly and adjustment of the optical system unit.

1. A nucleic acid analysis apparatus comprising:
a DNA chip interface capable of carrying a DNA chip having a plurality of measurement sites;
a light-emitting diode for irradiating said plurality of measurement sites with excitation light; and
a CCD camera capable of detecting fluorescence emitted from said plurality of measurement sites.

2. The nucleic acid analysis apparatus according to claim 1, wherein a single DNA chip is irradiated with excitation light emitted from a plurality of light-emitting diodes simultaneously.

3. The nucleic acid analysis apparatus according to claim 2, wherein the single DNA chip is irradiated with excitation light from a set of opposite light-emitting diodes each of which is spaced apart from said DNA chip by substantially the same distance, such that the angle formed by the optical axis of excitation light from each light-emitting diode and a plane including said plurality of measurement sites is substantially the same for both of said light-emitting diodes.

4. A nucleic acid analysis apparatus comprising:
a DNA chip interface capable of carrying a DNA chip having a plurality of measurement sites;
a light-emitting diode capable of irradiating said plurality of measurement sites with excitation light; and
an optical system unit comprising a CCD camera, a housing, and a filter member, said housing containing a group of lenses for focusing fluorescence emitted from said plurality of measurement sites on said CCD and comprising an opening provided in the side thereof, and said filter member comprising a plurality of optical filters for selecting a wavelength and having a part thereof inserted into said opening, wherein the optical filter that is inserted among said group of lenses is switched by moving said filter member through a through-hole provided in a part of said housing.

5. The nucleic acid analysis apparatus according to claim 4, wherein said optical system unit comprises a frame for carrying said CCD camera, said housing, and said filter member, wherein focusing is carried out by moving said frame while having said DNA chip immobilized.

6. The nucleic acid analysis apparatus according to claim 5, wherein said optical system unit repeats the transport of said frame and measurement of said CCD camera for focusing.
